(19) United States
(12) Patent Application Publication
(10) Pub. No.: US 2007/0207208 A1
(43) Pub. Date: Sep. 6, 2007

(54) Compositions and Methods Using a Cardiac Glycoside

(75) Inventor: Lawrence Solomon, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(73) Assignee: ACCU-BREAK Technologies, Inc.

(21) Appl. No.: 11/680,444

(22) Filed: Feb. 28, 2007

Related U.S. Application Data
(60) Provisional application No. 60/777,942, filed on Mar. 1, 2006.
(60) Provisional application No. 60/778,113, filed on Mar. 1, 2006.

(51) Int. Cl. A61K 31/704; A61K 9/20

(52) U.S. Cl. 424/464; 424/774; 514/26

(57) ABSTRACT

A method of treating patients suffering from renal failure and concomitant cardiovascular disease with a low dose of the cardiac glycoside known as digoxin, and novel dosage forms to accomplish the method and to perform other uses.

### CROSS-REFERENCE TO RELATED APPLICATIONS

This is a continuation in part of U.S. Provisional Patent Applications, Ser. Nos. 60/777,942 and 60/778,113 both filed Mar. 1, 2006.

### BACKGROUND OF THE INVENTION

(1) Field of the Invention

This invention is concerned with compositions and methods of treating patients using a cardiac glycoside, e.g., digoxin. The invention comprises administering a low dose of digoxin to a patient suffering from reduced creatinine clearance, renal insufficiency or chronic renal failure, and concomitant cardiovascular disease.

(2) Description of the Related Art

Cardiac glycosides are a class of drugs useful for the treatment of heart failure and irregular heartbeat. Digoxin, a glycoside obtained from Digitalis lanata and other sources, is the most commonly prescribed of the cardiac glycosides, and is marketed in the U.S. as Lanoxin® in 125 and 250 microgram (mcg) tablet strengths. Digoxin has been known for many years and, in the U.S., was marketed before the Food and Drug Administration (FDA) began regulating pharmaceuticals. Lanoxin®, a brand of digoxin tablets marketed in the U.S., was formally approved by the FDA in 1977 under a New Drug Application. Outside the U.S., 62.5 mcg digoxin strengths are marketed as unscored tablets. It is known to administer digoxin as a solid dosage form in doses of 62.5 mcg or more, in increments of 62.5 mcg.

It has been disclosed to provide a solution of digoxin in a soft gelatin capsule (Lanoxicaps®) in doses of 50, 100 and 200 mcg. In human patients, the absorption of the solution of digoxin from the commercial product into the blood is 90-100% of the provided dose (a “bioavailability” of about 90-100%). Absorption, or bioavailability, from a conventional solid dosage form of digoxin (e.g. a Lanoxin tablet) is 60-80% of the provided dose. The Lanoxicaps® label (Product Information), incorporated herein by reference (Physicians' Desk Reference (“PDR”), 2005 Ed., p. 1540) discloses a 50 mcg dose. The PDR expressly provides that the 50 mcg dose from a Lanoxicaps product is equivalent to a 62.5 mcg dose of a Lanoxin tablet. In addition, as a liquid-containing soft gelatin capsule, the Lanoxicaps product cannot be as readily divided, nor provide dosing flexibility as from a scored or otherwise breakable tablet. A 50 mcg/ml elixir is also approved in the U.S.

The Lanoxin Product (Prescribing) Information, available at page 1553, et seq. of the 2005 PDR and incorporated herein by reference, discloses that Lanoxin is useful for congestive heart failure (CHF) and chronic atrial fibrillation, and that dosage should be adjusted based on renal function and lean body weight. Table 5 at page 1556 of that PDR discloses the recommended doses for Lanoxin, reciting that 125 mcg/day is the minimum recommended maintenance dose except in the specific case of a 50 kg anuric patient (i.e., a patient with a corrected creatinine clearance (“CCr”) of zero). The recommended dose for a 60 kg person is 125 mcg per day. The recommended dose for a 50 kg person with a CCr of 10 (which is severely impaired) is also 125 mcg daily. When renal failure patients are dialyzed, the fraction of digoxin in the body that is removed by each dialysis is minimal.

In addition, the Lanoxin label states that patients with arial fibrillation generally require more digoxin than is needed for treatment of patients with CHF. It would thus be surprising that 75 mcg or less per day of digoxin could treat either CHF or atrial fibrillation, unless the patient had a body weight of about 50 kg and no urine output.

There is no known disclosure of administering digoxin at a dose less than 62.5 mcg. Nor does the prior art teach that the close of digoxin should be adjusted in dosage increments other than 62.5 mcg or whole multiples thereof per dose or per day.

The importance of what for most drugs is a modest therapeutic difference in dose is illustrated in the article, Association of Serum Digoxin Concentration and Outcomes in Patients with Heart Failure, JAMA Feb. 19, 2003-Vol 269, No. 7, pp 871-78 (Rathore S. et al). The authors demonstrate that, for example, a serum digoxin concentration of 0.72 (ng/ml) may be materially safer than one of 0.90 (see FIG. 3 therein). This is the same ratio between the dose of the current invention and the lowest disclosed dose of digoxin, which is 62.5 mcg. Thus there is substantial reason to adjust digoxin precisely. That need is especially great in patients who retain their administered dose for long periods of time, as is found in renal failure patients (including both the acute and the chronic types of renal failure).

As is the case with other highly potent drugs, content uniformity considerations lead digoxin to be produced as a small tablet. Typically, digoxin tablets are about 6 mm in diameter and about 3 mm in thickness, with higher does tablets larger than lower dose tablets believed that round tablets are preferred because providing the same volume or mass tablets in a different shape, such as oblong tablets, but having the same volume or mass as the current round tablet, would make them more fragile and prone to breakage. Providing them as substantially square shaped tablets with the same mass as the current tablets would give them have a longest dimension (length or width) less than that of the longest dimension (i.e., diameter) of the circular configuration. Accordingly, a square digoxin tablet of the same volume or mass as the commercially available round tablet would be less easily manipulated.

The 125 mcg and 250 mcg round digoxin tablets are scored, apparently to allow for tablet breaking, but it is generally accepted in the medical and pharmacy fields that breaking of currently available digoxin tablets of the size described above is difficult. Breaking of a currently available digoxin tablets can be especially difficult for the typical elderly patient in need of digoxin therapy. When broken by a typical user, digoxin tablets tend to result in inaccurately divided doses. Because of the occasional, even frequent, need to adjust the dose of digoxin in individual patients, the limited difference between therapeutic and toxic effects, and the current art that fails to provide a conveniently or accurately breakable digoxin tablet, it is therefore important to provide a digoxin dosage form which allows subdivision by most people to yield a predictable partial dose as desired.

The subject invention provides digoxin tablets that can overcome the problem of difficult or inaccurate breaking as occurs with currently available small, round tablets.

The availability of scored tablets in 125 mcg strengths provides flexibility, e.g., the enhanced ability to split the tablet into two parts, for patients who are treated with digoxin doses of 125 mcg/day and above. Because there is overlap between therapeutic digoxin doses and doses that cause bothersome or serious side effects, a need exists for a dosage form that allows similar flexibility of dosing at doses that are lower than 125 mcg/day.

### SUMMARY OF TILE INVENTION

The subject invention provides embodiments for a novel and advantageous dosage form containing a cardiac glycoside, preferably digoxin, as the active pharmaceutical ingredient (APT). The subject dosage form advantageously meets regulatory and commercial standards for friability, and is easily divisible into accurately divided doses when broken. Preferred embodiments of the dosage form of the subject invention are adapted to maintain their integrity during manufacture, shipping and handling, but are easily and accurately broken by a patient or caregiver when intentionally dividing the dose. The invention preferably provides a novel low-dose dosage form comprising a cardiac glycoside such as digoxin or digitoxin. Methods of treatment comprising administering a low daily dose of the cardiac glycoside are also included within the scope of this invention.

One preferred embodiment of the subject invention includes an elongated (e.g., capsule-shaped) tablet. For purposes of the subject invention, “elongated” refers to tablets that are taller than either cross-sectional dimension (“taller than wide” as defined in WO 2005/112897), and for tablets that are not taller than wide, “elongated” refers to tablets in which the width and the depth are not substantially identical (as defined in WO2005/112897).

Alternatively, tablets that are either scored deeply (“deep-scored”) or are provided with a plurality of scores (such as cross-scored) are also included as embodiments of the subject invention. For purposes of the subject invention, “deep-scored” means a tablet having a score which is greater than about 50% through at least one layer or segment of the tablet; “cross-scored” means a tablet having two intersecting score lines forming a “cross” or a “plus” configuration,

The subject dosage forms may be manufactured by compression in a conventional or a layer tablet press, or may be manufactured as adhesively bonded tablets, the latter such as in accordance with the disclosure of US 2006/0003000. More preferably, the subject dosage forms are embodied as segmented tablets, as described in WO 2005/112900 and PCT/US05/18632, WO 2005/112870, WO 2005/112897, and WO 2005/112898, in which at least one layer or segment comprises a cardiac glycoside such as digoxin. Pertinent portions from each of the above-referenced published patent applications are incorporated herein by reference.

One preferred embodiment of the invention involves an elongate layered tablet having at least two segments in which a first segment comprising digoxin has an outer (upper or lower as created in a typical tablet press) surface and an opposing face that contacts a second segment. This tablet thus further comprises a second segment having a contacting face in contact with the first, digoxin-containing (“active”) segment. Typically, the total amount of digoxin in all active segments is a therapeutic quantity. The first and second segments will generally also comprise one or more pharmaceutically acceptable excipients. The second segment is preferably free of API except for digoxin that has been inadvertently transferred to that segment during the manufacturing process and is most preferably therefore a pharmaceutically inactive composition. This second segment advantageously can provide structural strength to the first segment and to the tablet as a whole, and can serve as the segment through which optional tablet breaking can occur. It would be readily understood that each of the segments of the compositions described herein can be provided as a single layer or as more than one layer of substantially identical composition.

The second segment may be in contact with an additional active segment. For example, the first segment comprising digoxin may be a unitary segment (as defined in WO 2005/112900) and the tablet may therefore have a plurality of unitary segments adjoining the same side of the same second inactive segment described above. In a different embodiment, another preferred embodiment of the invention comprises a three-segment composition wherein a first and third segment each comprise an API, e.g., digoxin, and said second segment lacks a pharmacologically effective quantity of digoxin and is disposed between those first and third segments so that the first and third segments do not contact each other. Alternatively, one of the API-containing segments may comprise a different or an additional API than is present in the other API-containing segment. For example, a first segment can comprise digoxin as the API, and the third segment can comprise furosemide and/or another medicament commonly given to patients in need of digoxin. This third segment may also comprise digoxin.

Optionally, a marking or score may be provided in any of the segments of the dosage forms described above in order to guide or facilitate breaking of the dosage form at that specified location.

In another embodiment, the invention includes a scored tablet comprising about 120 mcg or less of a cardiac glycoside, and preferably comprising about 100 mcg or less of the cardiac glycoside. More preferably, the subject invention comprises a dosage form comprising a cardiac glycoside, preferably digoxin, having a dosage strength selected from strengths of 12.5, 25, 50, 75, or 100 mcg and more preferably in very low dosage strengths ranging from about 12.5 mcg to about 60 mcg. Digoxin tablets of the subject invention may be scored or unscored, and tablets containing about 62.5 mcg digoxin, and up to about 120 mcg digoxin are preferably scored.

The dosage form of the subject invention may preferably be a tablet composition for administering an active ingredient that is a cardiac glycoside to a patient, or may also be administered in a hard gelatin capsule in which digoxin and any excipients are present in solid form such as a powder. A preferred dosage form is a scored tablet which has about 100 mcg or less of digoxin which is readily divisible into half doses. More preferably, the dosage form of the subject invention is a tablet composition comprising about 50 mcg of digoxin, said dose optimally being readily divisible into half doses each containing approximately 25 mcg. Either a scored or unscored 50 mcg dosage form may be used. According to the invention, digoxin is administered enterally and preferably orally. It is preferred that the tablets of the subject invention provide characteristics relating to absorption of drug into the bloodstream (“bioavailability”) of the active ingredient similar to the absorption or bioavailability of Lanoxin tablets of similar strength.

The subject invention further relates to a novel method of treatment of a patient in need of treatment with a cardiac glycoside such as digoxin or digitoxin. More preferably, the subject invention concerns a method for the treatment of renal failure or renal insufficiency patients, such as chronic renal failure patients receiving dialysis, who require cardiac glycoside therapy. Generally, patients who are targeted for treatment according to the invention are adults weighing from about 50 to about 100 kg and have CCrs of between 0-40 mL/min/70 kg. One preferred embodiment of the subject method comprises enterally administering a solid dosage form containing about 60 mcg or less of digoxin. More preferably, the subject method includes said treatment of a patient who suffers from renal failure or has anuria or a corrected creatinine clearance of approximately zero.

Accordingly, it is a primary object of this invention to provide a method of treating patients suffering from renal failure who require digoxin therapy wherein a low dose of digoxin is utilized. The low-dose digoxin treatment method can preferably be carried out using a digoxin tablet provided in a dosage strength selected from 12.5, 25, 50, 75 or 100 mcg. More preferably, the tablet is readily divisible into two half-doses, for example comprising a score to facilitate breaking of the tablet.

In addition, it is a primary object of the invention to provide improved methods of administration of digoxin to patients with CCrs of or less than 40 ml/min/70 kg and a body weight of from about 50 kg to about or above 100 kg, as detailed below and as claimed.

Another object of the invention is the method of treatment of patients with renal insufficiency in a dose lower than the minimum stated in Table 5 of the above-referenced Lanoxin Product Information.

In disclosing the invention, the inventors are disclosing digoxin tablets with similar absorption characteristics (bioavailability) to Lanoxin.

### BRIEF DESCRIPTION OF THE DRAWINGS

FIG. 1 depicts a perspective view of an embodiment of an elongated, two segment, unscored tablet in accordance with the subject invention.

FIG. 2 depicts a perspective view of an embodiment of an elongated, two segment unscored tablet in accordance with the subject invention, the tablet marked at the middle, perpendicular to the tablet length, for guiding breakage.

FIG. 3 depicts a side view of an embodiment of an elongated, two segment tablet in accordance with the subject invention, the tablet having a score formed at the middle, perpendicular to the tablet length.

FIG. 4 depicts a side view of an embodiment of an elongated, two segment tablet in the middle, perpendicular to the tablet length.

FIG. 5 depicts a side view of an embodiment of an elongated, two segment tablet in accordance with the subject invention, the tablet having two scores, one each in opposing faces of the tablet at the middle, perpendicular to the tablet length.

FIG. 6 depicts a side view of an embodiment of an elongated, two segment tablet in accordance with the subject invention, the tablet having two parallel, eccentric scores formed in the same face, perpendicular to the tablet length.

FIG. 7 depicts a side view of an embodiment of an elongated, two segment tablet in accordance with the subject invention, the tablet having two eccentric scores, one each in opposing faces of the tablet away from the midline, perpendicular to the tablet length.

FIG. 8 depicts a side view of an embodiment of an elongated, two segment tablet in accordance with the subject invention, the tablet having a deep score formed into a single layer, at the middle, perpendicular to the tablet length.

FIG. 9 depicts a side view of an embodiment of an elongated, two segment tablet in accordance with the subject invention, the tablet having a deep score formed into both segments, at the middle, perpendicular to the tablet length.

FIG. 10 depicts a side view of an embodiment of an elongated, two segment tablet in accordance with the subject invention, the tablet having three scores in one face of the tablet, perpendicular to the tablet length.

FIG. 11 depicts a side view of an embodiment of an elongated, three segment, unscored tablet in accordance with the subject invention.

FIG. 12 depicts a side view of an embodiment of an elongated, tri-segmented tablet in accordance with the subject invention, the tablet having a score formed at the midline of the inactive interposed segment, perpendicular to the tablet length.

FIG. 13 depicts a round, two segment tablet configuration in accordance with the subject invention showing a single deep score formed therein.

FIG. 14 depicts a round, two segment scored tablet embodiment in accordance with the subject invention showing the scoring in a “cross” configuration.

FIG. 15 depicts a two segment rectangular, scored tablet embodiment in accordance with the subject invention showing the scoring in a “cross” configuration at the midlines of one face of the tablet.

FIG. 16 depicts a two segment rectangular, scored tablet embodiment in accordance with the subject invention showing the scoring in a “cross” configuration, corner-to-corner of one face of the tablet.

### DETAILED DESCRIPTION OF THE INVENTION

The subject invention comprises a dosage form comprising at least one cardiac glycoside as an active ingredient. Preferably, the cardiac glycoside used as the active ingredient in the dosage form of the subject invention is digoxin. A preferred embodiment of the subject dosage form is an elongated tablet that is convenient to break into smaller tablettes. Such breaking is often performed in order to adjust the dose of a medication provided to a patient.

A dosage form of the subject invention can be provided as any shape. Where the invention utilizes the current round shape, the invention involves either the use of a plurality of scores, such as those that trisect or quadrisect the tablet, or else provides a larger tablet than has up until now currently been produced. Square or triangular, including equilateral triangular-shaped, tablets are included as part of the invention. One preferred embodiment of the subject dosage form having a length (or height), width, and depth, comprises an elongated shape wherein the ratio of the longest dimension (e.g., length) to the next or second longest dimension (e.g., width) ranges from about 1.5:1 to about 3:1. It would be readily understood that a cylindrical dosage form would provide this ratio as length:diameter. Preferred lengths of the subject dosage forms for adult human use are about 8 mm to about 25 mm. A preferred width or diameter (or depth) of the subject dosage forms are about 3 mm to about 10 mm, though the above ratios and precise dimensions are not limiting.

Ingredients and methods for production of the digoxin drug products are known in the pharmaceutical arts. See, for example, Remington's Pharmaceutical Sciences 20th Ed., Mack Publishing Co., Easton, Pa. (2000), which is incorporated by reference, and describes ingredients and various techniques commonly utilized in making compressed tablets. Pharmaceutically acceptable ingredients for active and inactive compositions used in a tablet of the subject invention may include the same ingredients as in a currently marketed digoxin tablet, which are enumerated in the Product Information for Lanoxin, as disclosed in the Physicians' Desk Reference, 2006. Specific production methods for certain configurations of tablets of the invention are disclosed in the incorporated publications US 2006/0003000, WO 2005/112900, WO 2005/112870, WO 2005/112897, and WO 2005/112898.

Embodiments of the subject invention can be understood by reference to the accompanying drawings. FIG. 1 is a perspective view of an embodiment or an elongated bi-segmented (i.e., two segment), non-scored tablet 100 having one of its dimensions, e.g., length A-A′, which is substantially greater than its other dimensions e.g., width B-B′ or depth C-C′. The tablet comprises a first segment 101 containing active pharmaceutical ingredient (API), preferably a cardiac glycoside such as digoxin, and a second segment 102 which is a different composition than first segment 101. Preferably, second segment 102 is substantially free of the API in the first segment, and is more preferably substantially free of API, i.e., is an inactive segment. The configuration of tablet 100 is shown without a score or other guide for breaking the tablet into divided doses.

FIG. 2 illustrates an embodiment of an elongated, bi-segmented tablet 200. This embodiment is similar to that shown in FIG. 1, having an API-containing first segment 201 and a second segment 202 which is preferably an inactive segment. However, this embodiment is shown to include a marking 203 formed onto the surface of the tablet which can provide guidance to the user for breaking the tablet in the location or general vicinity of the marking. The marking can be an imprint, a color designation or other means for designating an area or location for breaking of the tablet, if desired, and can be formed during or after compression of the tablet. In this FIG. 2, the marking is shown as a line positioned at the midline of the length of the tablet, and intersecting both segments of the tablet. However, the location of the marking is not critical and can be placed at any position where breaking may be desired, and can be of any length, e.g., covering only a portion of one of the segments.

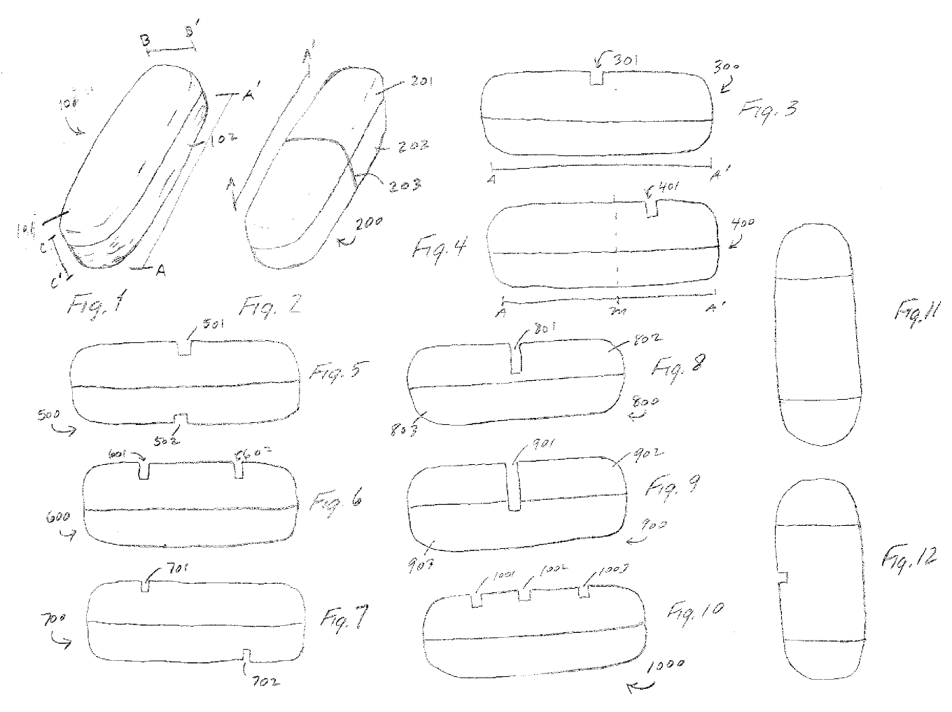

FIGS. 3-10 illustrate scoring patterns that may be used in accordance with the elongated bi-segmented tablets of the subject invention. For example, FIG. 3 shows a side view of an embodiment of an elongated tablet 300 having a score 301 of conventional depth formed at the midline, perpendicular to the tablet length A-A′. These scoring patterns described and shown herein for the tablets of the subject invention can be formed during manufacture of the tablet, e.g., by a conventional debossing protrusion provided in the tablet die, or can be made after tablet compression by removal or “de-massing” of tablet material by a cutting or other material-removing means, such as a laser, saw, or blade. FIG. 4 illustrates an embodiment of an elongated, bi-segmented tablet 400 having an eccentric score 401 formed away from the midline (shown as dotted line m), perpendicular to the tablet length A-A′.

FIGS. 5-7 illustrate varying configurations of elongated tablets having two scores formed therein. For example, FIG. 5 shows a side view of an elongated, bi-segmented tablet 500 having two scores 501 and 502, one each in opposing laces of the tablet at the midline, perpendicular to the tablet length. FIG. 6 shows an elongated, bi-segmented tablet 600 in accordance with the subject invention, the tablet having two parallel, eccentric scores 601 and 602, both formed in the same face of the tablet, perpendicular to the tablet length. FIG. 7 shows an elongated, bi-segmented tablet 700 having two eccentric scores 701 and 702, one each in opposing faces of the tablet, away from the midline and formed perpendicular to the tablet length.

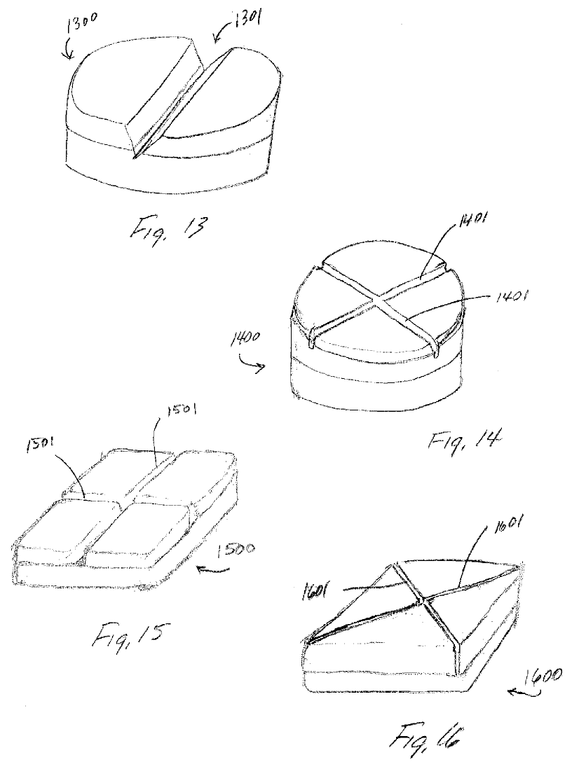

FIGS. 8 and 9 illustrate examples of configurations of a deep score formed into a tablet of the subject invention. FIG. 8 shows a side view of an elongated, bi-segmented tablet 800 having a deep score 801 formed only into a single segment 802 of the tablet, and is not formed into second segment 803. FIG. 9 is a side view of an elongated, bi-segmented tablet 900 having a deep score 901 formed completely through a first segment 902 and contacting second segment 903. The deep scores illustrated in FIGS. 8 and 9 show each score formed at the midline, perpendicular to the tablet length. However, it would be understood that the score may be formed eccentrically, as exemplified in FIG. 4. Moreover, a plurality of deep scores may be formed into a tablet of the subject invention, as exemplified in FIGS. 5-7 and FIG. 10.

FIG. 10 is shown to illustrate a bi-segmented tablet 1000, which has more than two scores (specifically three scores 1001, 1002, and 1003) formed into the tablet. These three-score embodiments may also vary in location, being formed at different positions in a single face of the tablet or in different faces of the tablet. In addition, the three-score configuration can include one or more deep scores.

FIGS. 11 and 12 show embodiments of elongated, tri-segmented tablets in accordance with the subject invention. FIG. 11 shows a non-scored tablet 1100 comprising three segments 1101, 1102, and 1103. In a preferred embodiment of the subject invention, segments 1101 and 1103 comprise an API-containing composition, and segment 1102 comprises a substantially inactive composition, i.e., substantially free of API. FIG. 12 is a side view of an elongated, tri-segmented tablet, similar to the tablet embodiment of FIG. 11, but having a score 1204 formed at the midline of the inactive segment 1202, which is interposed between APT-containing segments 1201 and 1203.

It should be readily apparent to persons of ordinary skill in the art that the subject elongated tablets are not limited to having two or three segments. Embodiments comprising four or more segments may be produced by equipment that is commercially available and in conventional use for manufacture of tablets having four, five, or more layers or segments. The number of layers or segments capable of being formed in tablets of the subject invention is limited only by the manufacturing equipment that is available.

FIGS. 13-16 illustrate additional embodiments that are considered to be part of the subject invention. For example. FIG. 13 shows a conventionally shaped (round), bi-segmented tablet 1300. However, this embodiment of the bi-segmented tablet of the subject invention includes a deep score 1301 formed therein. It is understood that the deep score can be formed into a single segment (as exemplified for the elongated tablet in FIG. 8, or can be formed into both segments (e.g., as shown in FIG. 9 for an elongated tablet). Additionally, the deep score can be a plurality of scores as exemplified for the elongated embodiments shown in FIGS. 5-7 and 10.

A further embodiment of the subject invention, shown in FIG. 14, comprises a scored bi-segmented round tablet 1400, wherein score 1401 is formed as a “cross” configuration, i.e., where the score is formed by lines that intersect at 90-degrees. This configuration can be formed in a single debossing step during compression or can be formed by making two or more lines in the compressed tablet by removing material (“de-massing”) after compression of the tablet. The cross configuration scoring for the round, bi-segmented tablet can be of conventional depth or can be a deep score into one or both of the segments.

FIG. 15 shows one embodiment of a rectangular, bi-segmented scored tablet 1500. Score 1501 is a “cross” configuration, as formed at the midlines in a face of one segment of the tablet. A different configuration for the “cross” scoring pattern useful in a rectangular bi-segmented tablet is shown in FIG. 16, wherein a bi-segmented rectangular, scored tablet 1600 comprises a score 1601 formed therein, the score extending from corner to corner of one face of the tablet. This scoring configuration can be formed as a score of conventional depth or as a deep score.

Certain embodiments of the present invention utilize a preferably scored tablet containing about 12.5 to 60 mcg of digoxin. One benefit of the invention is the case in which incremental dose adjustments may be calculated by patients, physicians and nurses. It is well known to physicians that patients have difficulty in calculating the numerical contents of a part of a solid dosage form when carrying out instructions to modify a dosage regimen that uses increments of the 62.5 mcg dosage unit, especially by dividing 125 mcg by two. The use of a dosage form that allows for incremental adjustment using 25 mcg increments provides a simplified means of achieving dosage adjustment by specifying dose adjustments from 50 to 75 mcg, or from doubling said dose to 100 mcg, rather than in making an analogous adjustment of dosages from a starting dose of 62.5 mcg. In addition, halving the dose to 25 mcg rather than to 31.25 mcg is more readily calculated.

A breakable 50 mcg tablet simplifies the daily administration of digoxin should the desired dose be below 50 mcg. For example, a regimen of administering 25 mcg per day on one day and 50 mcg or more per day on another day may be utilized. A specific example of this preferred dosing regimen includes alternating doses of 25 mcg/day and 50 mcg/day, which may benefit certain patients such as dialysis patients.

It is further observed that one of the main side effects of digoxin therapy is nausea, which is also a common side effect of the uremic (i.e., anuric) state. The ability to tailor digoxin therapy optimally to the uremic/anuric patient based on individual tolerability, and thereby advantageously reduce the incidence or severity of nausea caused by higher doses of digoxin, is provided by the invention.

Another advantage of the invention is the ability to provide a digoxin dose in the above-mentioned increments suitable for accurate adjustment of digoxin blood levels or maintaining levels within a more narrow desired range. Measurement of said levels is an important part of modern evaluation of digoxin therapy, especially when signs and symptoms suggesting either toxicity or ineffectiveness occur. The prescribing information for digoxin discloses that patients taking a single administration per day of digoxin have less reliable measurements of blood levels when blood is drawn six to eight hours after the last dose than do patients who split their daily digoxin ingestion into two doses. Thus, the invention allows a renal failure patient having digoxin levels measured to take, for example, a half of a 50 mcg dose twice daily on the day of the measurement, while having the convenience of taking a whole tablet 50 mcg tablet once daily on days when blood levels are not being measured.

The invention also provides a method of treating a patient with anuria who is in need of digoxin therapy. This method comprises enterally, and preferably orally, administering to a said anuric patient weighing approximately 40-100 kg., a daily dose of 25, 50, 75 or 100 mcg of digoxin, or different daily doses in increments of those doses. This method also extends to patients suffering from congestive heart failure and chronic atrial fibrillation who require a slowing of the ventricular heart rate.

An additional feature of the invention is a method of treating a patient in need of digoxin therapy who has a creatinine clearance of about 10 mL/min/70 Kg and a weight of from about 40 to about 100 kg which comprises enterally administering an effective amount of digoxin in the range of from 50-75 mcg per day. This method also extends to patients suffering from congestive heart failure and chronic atrial fibrillation with an undesirably rapid ventricular response who require a slowing of the ventricular response rate.

The invention comprises a 50 mcg digoxin dosage form which can be formulated with suitable excipients. Optimally, the invention is a tablet and more optimally a breakable tablet such as a scored tablet.

To implement the invention, a digoxin solid dosage form, preferably a scored tablet is produced that, on a dosage weight-weight basis, can be substantially bioequivalent to Lanoxin tablets. The marketing of several tablet products that are bioequivalent to Lanoxin in the U.S. demonstrates that such is feasible. Pharmaceutically acceptable excipients may be utilized.

Methods of administration of the invention may in a preferred example be utilized by a patient with an approximate CCr, preferably connected, of from 5 and 50 mL/min/70 kg and a weight of about 40 to about 100 kg or from 50-60 kg., who is receiving dialysis treatments and suffers from CHF, and who is taught by the Lanoxin product in Formation to receive a maintenance digoxin dose of 125 mcg per day. Patients having a CCr of 20 or from 30-40 mL/min/70 kg will benefit from this inventive method of treatment. In the invention, said patient, after an optional period of a loading dose that typically comprises a higher than maintenance dose schedule, receives 50 mcg/day for at least two weeks. The clinical response and possibly SDC are evaluated and as needed, the dose is adjusted. Said adjustment may be to 1½ tablets daily, though it is possible that a dosage of less than 50 mcg/day could be appropriate. After at least another two weeks, the dosage is re-evaluated.

If the patient has been receiving 75 mcg/day in either one or two daily doses but a higher dose is needed, then a dose of 100 mcg/day, in either one or two daily doses, is indicated. The majority of such patients will be successfully treated with this method, and thus will minimize the risk of toxicity that could come from being given 125 mcg of digoxin daily, as would be the current teaching.

Those skilled in the art will recognize that while specific embodiments have been described herein, various modifications may be made without departing from the scope of the invention. Other embodiments and/or uses of the invention will be apparent to those skilled in the art from this disclosure and the scope of the invention is intended to be limited only by the following claims.

1. A method of treating a human patient in need of treatment with a cardiac glycoside, said method comprising enterally administering a solid dosage form containing about 60 mcg or less of said cardiac glycoside selected from the group consisting of digoxin and digitoxin.

2. The method of claim 1 wherein said patient suffers from renal failure.

3. The method of claim 1 in which said dosage form is ingested orally.

4. The method of claim 1 in which said dosage form comprises a tablet.

5. The method of claim 4 in which said tablet is scored.

6. A method of treatment using digoxin, said method comprising administering to a person in need of digoxin a daily dose of digoxin, said daily dose selected from the group consisting of about 12.5, 25, 50, 75, and 100 mcg.

7. The method of treatment as defined in claim 6 in which said dose is provided as a tablet or as an encapsulated tablet.

8. The method of treatment as defined in claim 7, said method comprising administering a whole or half of a 50 mcg tablet or encapsulated tablet.

9. The method of treatment as defined in claim 8 in which the patient suffers from chronic atrial fibrillation with all undesirably rapid ventricular response rate.

10. The method of treatment defined in claim 8 in which said patient suffers from congestive heart failure.

11. The method of claim 6 wherein said patient has anuria or a corrected creatinine clearance of approximately zero.

12. A method of administering digoxin to a patient in need of digoxin therapy who has a corrected creatinine clearance of about 5-50 mL/min/70 kg and who weighs about 40-100 kg or less, said method comprising enterally administering about 50 to about 100 mcg of digoxin daily in doses of 25 mcg or a whole multiple thereof.

13. The method of administering digoxin as defined in claim 12 in which said patient suffers from congestive heart failure.

14. The method of administering digoxin as defined in claim 12 in which said patient suffers from atrial fibrillation with an undesirably rapid ventricular response.

15. The method of administering digoxin as defined in claim 12 in which the corrected creatinine clearance is about 30-40 mL/min/70 kg and the patient's weight is about 50-60 kg.

16. A pharmaceutical tablet comprising about 120 mcg or less of digoxin as an active pharmaceutical ingredient, wherein said tablet is scored when the dosage strength of said digoxin in the tablet is between, and inclusive of about 60 mcg and about 120 mcg.

17. The pharmaceutical tablet of claim 16 wherein said tablet is scored when the dosage strength is less than about 60 mcg.

18. The pharmaceutical tablet of claim 16, said tablet comprising about 25 mcg of digoxin as an active pharmaceutical ingredient.

19. A pharmaceutical dosage form comprising a cardiac glycoside, said dosage form being breakable into divided doses, said dosage form comprising:
a) a tablet formed from a single layer that has other than a circular dimension; or
b) a plurality of layers or segments; or
c) an elongated shape having a longest dimension and a second-longest dimension, and a ratio of the longest dimension to the second longest dimension preferably of about 1.5:1 to about 3:1; or
d) a score selected from a deep score and a pattern comprising a plurality of scores; or.
e) a height or width of greater than about 8 mm.

20. The pharmaceutical dosage form of claim 19 wherein said dosage form comprises a tablet.

21. The pharmaceutical dosage form of claim 19 wherein the cardiac glycoside is digoxin.

22. The pharmaceutical dosage form of claim 21, wherein said dosage form comprises about 25 micrograms to about 500 micrograms of digoxin.

23. The dosage form of claim 19, said dosage form being an elongated tablet.

24. The dosage form of claim 19, said dosage form comprising a quadrisecting score pattern, said pattern selected from the group consisting of a cross-scored pattern and the pattern comprising three parallel scores.

25. The pharmaceutical dosage form of claim 19, said dosage form comprising a plurality of segments wherein a first segment is pharmacologically active and a second segment is pharmacologically inactive.

26. The pharmaceutical dosage form of claim 25 that comprises a tablet consisting essentially of one segment comprising digoxin and one segment that is pharmacologically inactive.

27. The pharmaceutical dosage form of claim 25 having a first pharmacologically inactive segment interposed between an upper active segment and a lower active segment, wherein at least one of said upper and lower active segments comprises digoxin.

28. The pharmaceutical dosage form of claim 25 wherein said segmented dosage form is scored.

29. The pharmaceutical dosage form of claim 19 in which the dosage form further comprises a pharmacologically effective quantity of a drug other than a cardiac glycoside.

30. The pharmaceutical dosage form, of claim 27 in which one segment comprises a pharmacologically effective quantity of a cardiac glycoside and a different segment comprises a pharmacologically effective dose of a different drug.

31. The pharmaceutical dosage form of claim 19 in which the ratio of the height to the width or the width to the depth of the tablet is from about 2:1 to about 2.5:1.

32. The pharmaceutical dosage form of claim 19 in which said tablet is substantially homogeneous.

33. The pharmaceutical dosage form of claim 19 comprising a first segment containing a therapeutic quantity of digoxin and a second segment comprising a lesser quantity of digoxin.

34. The pharmaceutical dosage form of claim 19 containing an amount of digoxin in a range selected from the group consisting of from 10-750 mcg., from 25 to 500 mcg, and from 50 to 500 mcg.

35. The pharmaceutical dosage form of claim 19 wherein said dosage form has a printed mark designating a desired breaking region of said dosage form.

36. A method for treating a patient in need of cardiac glycoside therapy, said method comprising:
providing a cardiac glycoside pharmaceutical dosage form as a substantially elongated tablet or having a deep score, said dosage form being readily breakable into a plurality of tablettes containing predictable quantities of said cardiac glycoside.

37. The method of claim 36, said elongated tablet having a longest dimension and a second-longest dimension, and a ratio of the longest dimension to the second longest dimension of about 1.5:1 to about 3:1.

38. The method of claim 36 wherein said cardiac glycoside is digoxin.
